(19) United States
(12) Patent Application Publication
(10) Pub. No.: US 2015/0072413 A1
(43) Pub. Date: Mar. 12, 2015

(54) CELL CULTURE APPARATUS AND CULTURE METHODS USING SAME

(71) Applicants: ARIZONA BOARD OF REGENTS ON BEHALF UNIVERSITY OF ARIZONA, Tucson, AZ (US); UNIVERSITÉ DU LUXEMBOURG, Luxembourg City (LU); CENTRE DE RECHERCHE PUBLIC-GABRIEL LIPPMANN, Belvaux (LU)

(72) Inventors: Frederic Zenhausern, Fountain Hills, AZ (US); Matthew Estes, Chandler, AZ (US); Paul Wilmes, Bettembourg (LU); Pranjul Shah, Esch-sur-alzette (LU)

(21) Appl. No.: 14/389,137

(22) PCT Filed: Mar. 27, 2013
(86) PCT No.: PCT/EP2013/056607

§ 371 Date: Sep. 29, 2014

Related U.S. Application Data
(60) Provisional application No. 61/617,393, filed on Mar. 29, 2012.

(51) Int. Cl. C12M 1/12; B32B 38/18; B32B 37/12; C12M 3/06; C12N 5/09

(52) U.S. Cl. CPC C12M 25/02; C12M 23/16; C12N 5/0693; B32B 37/1292; B32B 38/1841; C12N 2502/70; B32B 2307/726; B32B 2369/00
USPC 435/347; 435/297.4; 156/291

(57) ABSTRACT

Cell culture apparatus comprising at least two adjacent cell cultivation channels separated by a permeable or semipermeable membrane, wherein at least one channel, for the majority of its length, has a cross sectional area of no more than 1 mm², said channel being provided with entrance and exit means to permit the passage of media therethrough, allows co-culture of separate cell types, e.g. human and microbial cells, without mingling, allowing monitoring of cell cultures and chemical exchanges between the respective cell cultures.

### FIELD OF THE INVENTION

The present invention relates to cell culture apparatus and cell culture methods using the same.

### BACKGROUND OF THE INVENTION

Mixed microbial communities play pivotal roles in governing health and disease. At present, little is known about the underlying molecular and ecological processes that determine microbial and human cellular transitions between health and disease states. Recent evidence suggests that some diseases are mediated by microbial community disequilibria rather than being caused by single pathogenic strains. For example, the aetiology of certain idiopathic medical conditions, e.g. cardiovascular disease, diabetes or Parkinson's disease, has recently been linked to human gastrointestinal microbiota. However, at present, causative links are difficult to ascertain, primarily owing to a lack of in vitro human-microbial co-culture systems which allow prolonged co-culture and in which emergent hypotheses can be tested. Simple co-culturing of human and microbial cells is not effective, owing to the pronounced differences in their respective growth rates, with microbial cells rapidly out-competing human cells in a standard culture situation.

Microbial communities associated with the human body play essential roles in the host's health by making allochthonous indigestible compounds bioavailable [Hooper et al., 2002; van Duynhoven et al., 2011], outcompeting pathogens [Donoghue, 1990], regulating angiogenesis [Stappenbeck et al., 2002], ensuring proper enteric nerve function [Husebye et al., 1994], influencing the central nervous system [Ochoa-Repáraz et al., 2011], and educating and maintaining the host's immune system [Macpherson and Harris, 2004; Artis, 2008; Round and Mazmanian, 2009; Chervonsky, 2010]. Consequently, humans have to be thought of as superorganisms or human-microbe hybrids [Goodacre, 2007]. The human intestinal microbiome alone contains at least 100 times as many unique genes as the human genome [Gill et al., 2006], and this microbial gene pool is highly adapted. However, not only do the intestinal microbiota provide beneficial genetic traits to the human host, e.g. for digestion [Hehemann et al., 2010], but are also involved in the production of metabolites that contribute significantly towards pathogenesis [Wang et al., 2011]. Consequently, the molecular interactions related to human and microbial mutualism, commensualism, and parasitism are in constant flux and there is currently great interest in determining the (eco-)system-level transitions to particular attractor states which reflect either human health or disease.

The aetiology of numerous idiopathic medical conditions, e.g. cardiovascular disease [Sandek et al., 2008; Wang et al., 2011], colorectal cancer (recently reviewed by [Candela et al., 2010], gastric cancer [Polk and Peek, 2010], Crohn's disease [Manichanh et al., 2006; Frank et al., 2007; Dicksved et al., 2008], obesity [Turnbaugh et al., 2006], type 1 [Wen et al., 2008; Giongo et al., 2011; King and Sarvetnick, 2011] and type 2 [Vrieze et al., 2010] diabetes, or even Parkinson's disease [Braak et al., 2006; Lebouvier et al., 2010; Shannon et al., 2010] has been linked to microbially driven disequilibria (dysbiosis) in the human gastrointestinal tract. Such links, albeit putative, have in the majority of cases only been possible to establish recently because of the application of high-resolution molecular methods to human microbial communities. Such tools involve in-depth microbial community profiling based on rRNA genes sequences (e.g. [Andersson et al., 2008]), community- or meta-genomics [e.g. Qin et al., 2010], metatranscriptomics [e.g. Gosalbes et al., 2011], metaproteomics [Wilmes, 2011], and (meta)metabolomics [e.g. Jansson et al., 2009].

The main advantage of high-resolution molecular approaches is that they are able to comprehensively probe microbial communities in situ. This is in direct contrast to traditional medical microbiological characterisation efforts based on the Henle-Koch postulates that rely on laboratory-based isolation of pure clonal pathogenic strains. Such reductionist approaches may prove futile for elucidating microbial community-mediated disorders because they do not allow the study of infectious agents in the full community context, a need that is reflected by the fact that current estimates predict that 99% of all microbial species cannot be obtained in axenic culture [Schloss and Handelsman, 2005]. Such approaches do not permit the diagnosis of, or allow the personalised treatment of microbial dysbiosis-driven diseases.

Although high-resolution molecular tools hold great promise for ascertaining specific links between certain microbial species and/or molecules, and human pathobiology, in vivo and in vitro models are required for answering unresolved fundamental questions related to human-microbial molecular interactions and for testing specific hypotheses. In vivo gnotobiotic animal models, which allow the direct manipulation of microbial community structure, environmental conditions, host genotype and other factors, have proven very successful for answering fundamental questions related to host-microbe molecular interactions and their links to pathophysiology [e.g. Turnbaugh et al., 2006] and for providing answers to questions arising from high-resolution molecular investigations on human subjects [Wang et al., 2011].

In vitro models of the human gastrointestinal tract have primarily been developed to simulate metabolic transformations in the human gastrointestinal microbiota [Macfarlane and Macfarlane, 2007]. These models incorporate mixed microbial communities derived from faecal inoculate and range from simple batch fermentation systems to more or less sophisticated, well-controlled, single or multi-stage continuous bioreactor systems [Macfarlane and Macfarlane, 2007]. While these systems have proven to be adequate functional models of gastrointestinal digestion processes, two recent publications which focused on the microbial community composition of two well-established models, the TIM-2 model [Minekus et al., 1999] and simulator of the human intestinal microbial ecosystem (SHIME; [Molly et al., 1993; Possemiers et al., 2004]), found significant differences between the expected microbial community and the microbial community that was actually established in the respective bioreactor vessels [Rajilic-Stojanovic et al., 2010; van den Abbeele et al., 2010], suggesting that these systems are not capable of reliably promoting the establishment of mixed microbial communities representative of the different parts of the gastrointestinal tract. Van der Abbeele et al., (2010) found that differences in the community structure may be related to the lack of mucosal surface in the SHIME model.

Current in vitro mixed culture gastrointestinal models do not include human cells and, hence, a major component is not present in current models, and it is not possible to establish whether this absence has a significant influence on the microbial communities that establish in the respective bioreactor compartments. The integrated co-culture of human and microbial cells should allow a more representative simulation of gastrointestinal metabolic processes, e.g. digestion of bioactive plant compounds, which are currently only simulated separately or consecutively using either human cell lines [Sergent et al., 2008; Biehler and Bohn, 2010] or microbial cultures [Go\ddot{n}i et al., 2006; Déat et al., 2009].

There is currently pronounced interest in developing microfluidics-based, in vitro model systems for the human gastrointestinal tract [Turnbaugh et al., 2007]. In vitro (micro-)fluidics-based systems have so far been mainly used for studying medically relevant biofilm formation within microbial isolate cultures [McBain et al., 2009; Coenye and Nelis, 2010; Saleh-Lakha and Trevors, 2010]. Although several research groups have co-cultured different human cell types [e.g. Bhatia et al., 1999; Stybayeva et al., 2009], only a limited number of studies have reported the successful co-culture of human and microbial isolates [e.g. Linden et al., 2007; PeRicanò et al., 2008; Subbiandoss et al., 2009; Saldarriaga Fernandez et al., 2011]. Recently, the potential of microfluidics-based approaches for devising human and microbial co-culture systems has been demonstrated in a study focused on host-pathogen interactions [Kim et al., 2010], and by the successful co-cultivation of symbiotic microbial communities in aqueous micro-droplets which were probed for synergistic interactions [Park et al., 2011].

A system that is designed to mimic the human gut is disclosed by Kim, H. J., et al., Lab On A Chip (http://pubs.rsc.org/en/content/articlelanding/2012/lc/c2lc40074j), and comprises two microfluidic channels separated by a porous flexible membrane coated with extracellular matrix (ECM) and lined by human intestinal epithelial (Caco-2) cells. The basal channel is used for the supply of nutrients. The construction material used in this system is a permeable plastic, polydimethylsiloxane. The drawbacks of this system are that:

1) Only probiotic, non-pathogenic microorganisms can be cultured, as the two cultures are grown together in one of the two culture microchannels. Consequently, no directly sampled microbial communities can be grown using this system because normal microbiota will rapidly outcompete and overgrow the human cells and/or cause direct human cell lysis due to presence of pathogenic bacteria and/or viruses;

2) Because the cell populations are mixed, no standardised or optimised growth conditions are achievable for either cell population;

3) It is not possible to measure the effects of pathogenic microorganisms in this culture model other than over very short timeframes;

4) It is not possible to co-culture >60% of human microbiota that are strictly anaerobic;

5) It is not possible to measure the effect of other human cell types/cultures without also mixing such cell types into the culture;

6) By essentially culturing mixtures of human and microbial cells, it is not possible to identify certain molecules originating from within specific cell populations; and

7) The absence of mucin or mucus producing cell lines means that the mucosal layer which plays an essential role in health versus disease states by controlling inflammatory processes is not modelled.

Ideally, a co-culture system would allow for the assembly, interaction and assay of human and microbial components to elucidate molecular, cellular and/or ecological networks that might affect health and disease states. More particularly, it would be desirable to provide:
1) The ability to co-culture both human and microbial cell populations in standardised and/or conditions for prolonged periods;
2) The ability to study pathogens in co-culture with human cells over in vivo relevant timeframes;
3) The ability to simulate anaerobic conditions present in the gut, thereby creating conditions mimicking those encountered by gastrointestinal microbial communities in vivo;
4) The ability to add additional cell populations into the model, e.g. different human cell lines, while still being able to provide standardised cultivation conditions;
5) The ability to relate individual molecules back to the cell populations of origin; and
6) The ability, by comprehensively mimicking in vivo conditions, to sustain microbial dysbiotic cultures and use these for diagnostic and tailored therapeutic purposes.

Flask and trans-well culture apparatus are standard cell-culture apparatus that cannot provide close proximity co-culturing of multiple cell lines or separate media supplies thereto. Existing techniques for microfluidic co-localisation prior to co-culture [Taff et al., 2007, Kim et al., 2009, Park et al., 2009, Ma et al., 2010, Frimat et al., 2011, Tumarkin et al., 2011] have allowed culture of various cell types in close proximity, but once co-culture is initiated, these approaches are unable to preserve distinct media supplies to the various cell types.

There is a need for apparatus that permits standardised and prolonged co-culturing of a plurality of cell lines/types that are physically separated but in chemical communication, with the possibility of separate media being supplied to each culture.

It has now surprisingly been discovered that it is possible to use the principles of microfluidics to provide adjacent culture channels separated by a permeable or semipermeable membrane that permit co-culture of separate microbial colonies with separate nutrient supplies, while allowing chemical interaction between the two channels.

### SUMMARY OF THE INVENTION

Accordingly, in a first aspect, there is provided culture apparatus comprising at least two adjacent cell cultivation channels separated by a permeable or semipermeable membrane, wherein at least one channel, for the majority of its length, has a cross sectional area of no more than 1 mm², said channel being provided with entrance and exit means to permit the passage of media through at least a portion of the channel having a cross sectional area of no more than 1 mm².

The culture apparatus of the invention may be made of any suitable material or materials, such as biocompatible glass, plastic substrate, including hard and soft polymers, hybrid organic and inorganic materials or ceramics and may be permeable or impermeable to oxygen, as desired. Composite and multilayer materials may be used, such as to provide structural integrity but with surfaces suited to cellular adhesion, or the whole may be made of a suitable, biocompatible, rigid plastic, preferably one that is not toxic, or not substantially toxic to the cells being cultured. A preferred plastics material is polycarbonate, or polystyrene that has typically been made wettable by oxidation.

In one aspect, the materials from which the apparatus is constructed may be further coated. Coatings may be applied as layers, such as insulating or conducting materials, including polymers, that can be deposited by techniques including electro-deposition and chemical vapour deposition (CVD). The surfaces of the materials may also be modified, such as by processing to form physical features ranging in sizes from nanometres to centimetres. Such features may provide controlled corrugation suitable for purposes of biomolecular interactions, for example cellular alignment, or may be adapted to create microenvironments with physico-chemical conditions to facilitate or lead to improvements in co-culture conditions of a species, or communities of species, such as by optimising chemical communication or spatial distribution between the biomolecular components and/or nutrients or other culture reagents or metabolites.

The channels may be provided by any suitable means, including targeted laser evaporation or guided, heated boring apparatus, but the provision of a membrane between channels established in this manner can prove difficult, although this can be achieved by leaving a thin wall between the two channels.

More preferred is to construct the apparatus in layers and to sandwich a suitable membrane between respective channels. For example, two channel-containing layers may be provided, each having a channel provided in one surface, a groove in the surface defining three sides of the channel, or as many sides as desired, but leaving one side open. When the channel-containing sides of the layers are brought together, they can be brought together such that the channels are in register. A membrane-containing layer may be located between the two channel-containing layers, thereby to locate a membrane between the two channels, the membrane defining the final side of each channel. It will be appreciated that this process may be suitably modified to accommodate multiple, adjacent channels. If there are more than two channels, any channel flanked by two or more channels will have open sides that can be completed by matching to a further channel and sandwiching a membrane-containing layer. A layer containing a channel that is flanked by two other channels may typically be a layer that has the thickness of the channel it defines and wherein the channel is a slot cut in the layer.

The thickness of the layers may be uniform or contain protrusions and/or recesses such as may be used to assist in engaging the other layers with which they are intended to interact. There is no limit to the shapes that may be used, and it is possible that a protrusion may pass completely through a hole in a middle layer to engage with a hole in a third layer, for example.

The membrane-containing layer may consist entirely of membrane material provided as a membrane, and preferably suitably tensioned until secured between the channel-containing layers, or may comprise a suitable web, matrix or lattice supporting the membrane prior to sandwiching. Such web, lattice or matrix may be removed after the membrane has been sandwiched, but it is generally preferable to leave it as part of the apparatus.

The membrane may be secured to either or both of the channel containing layers with which it interacts by any suitable means. Clamping may be used, but it is preferred to use an adhesive, or to cause the membrane to adhere to the channel-containing layers. The latter may be effected by sonication when one or both of the membrane layer and channel containing layer are formed from compatible materials. Suitable adhesives for plastics are well known in the art, but are less preferred owing to the accuracy required for the dimensions involved. Particularly preferred methods of adhesion are thermo-adhesion and pressure sensitive adhesives. In the former, the construct is heated by irradiation, or in an oven to cause at least one plastics material in the apparatus to become sufficiently tacky to adhere to an abutting layer. In one preferred embodiment, the membrane is supported by a ring of resiliently flexible material, such as a non-corrosive metal, rubber, or plastic, which serves to tension the membrane, thereby allowing the layers to be clamped thereon. The ring may also be non-circular, and even irregular, although a generally circular support is preferred to ensure an even tension on the membrane. Such membranes have the advantage of allowing easy removal of a layer and providing subsequent ready access to culture residing on the membrane.

The membrane may be permeable or semipermeable as required by the skilled person. It is preferred that the membrane does not permit passage of cells from one channel into another channel, otherwise the membrane may be selected such as to permit all molecules to freely pass between channels, or to more selectively permit passage. This may be achieved by providing suitably selected pores, such as ionic filters, hydrophobic, hydrophilic, or size filters. Semipermeable membranes are those which provide selective permeability for other than size of the molecules, organisms or viruses that can pass across the membrane. Semipermeable membranes are preferred. The membrane can also be formed by an assembly of fibres using a variety of materials and processing methods, including, for example, electro- and force-spinning methods. Embedded electromagnetic functions, such as electronic, optical and/or magnetic functions, may also be incorporated during the assembly or manufacture of the membrane.

The channels separated by a membrane preferably both, or all, have a majority of their length with a cross-sectional area of at least 1 nm², and preferably no more than 1 mm². It is generally preferable that the smaller dimension of the cross section of at least one channel is no more than 500 μm in that portion having a cross sectional area not exceeding 1 mm², and preferably for all channels having a cross sectional area not exceeding 1 mm². This is to take advantage of mass transport and microfluidic properties at the miniaturised scale, as well as other interactions known to occur in microfluidics, which, without being bound by theory, allows the flow of fluids in restricted diameter channels, with laminar flow and reduced Reynold's number, together with any other physico-chemical properties suitable for optimising the chemical communication and spatial distribution of the various biological species present within the device of the present invention.

In one aspect of the present invention, there is provided apparatus as defined, wherein the at least one cell cultivation channel has a cross section for a majority of its length that has two dimensions, and wherein at least one dimension does not exceed 500 μm. The second dimension may range from 100 nm to 5 mm, but is preferably no more than 2 mm.

The channels preferably have a uniform cross section for their entire length, or substantially their entire length between entrance and exit means, in order to permit through-flow of any media, whether even, interrupted or peristaltic, for example. The entrance means and exit means may simply be holes in the material defining the channels or chambers, or may comprise structures for affixing suitable pump means, or other actuator, sensor, or system for mass transport. For example, the entrance and/or exit means may comprise a nipple onto which may fit a tube from a pump.

The channels may be provided in any configuration desired, such as straight, serpentine, or circular, for example. Straight channels may be employed where multiple experiments are desired to be carried out, and the sets of adjacent channels may be provided in side by side arrangement in an elongate panel, for example. Three dimensional arrangements are also contemplated by the present invention.

In one preferred embodiment, the channels take the form of a swirl, or paired helix, in a form that might be obtained by drawing in a length by rotating the centre, and as is illustrated in accompanying FIG. 1, in which 10 is the apparatus, 20 is the entry means, 30 is the paired spiral channels, and 40 is the exit means. This assists in maximising the length of the channels while using a minimum of space. In this configuration, it is preferred that the entrance and exit points are located at the outer ends of the swirl. If the apparatus is intended for stacking, then the entrance and exit points may be located on protrusions or tongues, or may be in a side of the apparatus to allow access when stacked. In one embodiment, multiple apparatus are stacked and in serial communication from adjacent exit and entrance points located on opposite sides of each apparatus, thereby to easily stack multiple devices where the inlet of the lower layer mates or is otherwise in fluid communication with the outlet of the upper layer, such as by a luer type mating connection.

The entrance points, or means, may permit or comprise a plurality of media pumps, such as micropumps, or injection apparatus. These may be continuous, discontinuous, or peristaltic, and may be arranged such that, none, one, or more is active at any given time. When modelling specific systems, such as the human gut, it may be desirable to control the pumps such as to provide any desired level of complexity and highly controlled pumping protocols, especially where a plurality of apparatus units is connected in series, for example. In a preferred aspect, the pumps are controlled by one or more algorithms, such as bya controllable programmable software algorithm in combination with a computer.

The nature of the media to be pumped through the channels is any that is deemed appropriate by one skilled in the art, and may be a liquid or a gas, or a gaseous liquid, an amorphous liquid or the like, and may comprise nutrients, markers, reagent, ligand, solvent or any other substance that it is desired to pass through the channels or expose the contents of the channel to.

It is generally envisaged that at least one channel will be used to culture cells, such as animal, preferably human, cells, or microbes obtained from an animal or human. An adjacent channel may be used for a further cell culture, for example from a tissue or organ, or may be used for media, with or without cells. In one embodiment, three channels are separated in series by membranes, with media in a first channel, human intestinal epithelial cells, for example, adjacent thereto in a second channel, and a third channel being adjacent to the second channel and containing, for example, mixed microbial cultures from a target intestine, or may be a single microbial isolate. In other embodiments, nervous cells, immune cells or other biological assemblies may also be placed in at least one additional channel, such as may be located basally to the epithelial cell culture chamber.

It will be appreciated that multiple channels separated by membranes may be provided, and that the nature of the channel may be selected in accordance with the intended use, such as nutrient or cell culture, or all channels may be adapted for cell culture, for example, but may be used for other purposes, if desired. In a further embodiment, a media perfusion channel containing none, one or more other cell types, such as immune cells, or there may be provided a stack with further additional channels containing other additional cell types, such as neurons.

As used herein, the term “cell culture” in relation to a channel of the apparatus, as well as associated terms, refers to a culture of a microorganism, such as a single, preferably eukaryotic, cell type, or cell community, such as a tissue, adhered, preferably as a monolayer or consortium, on one or more walls of the channel, and may include pure isolates and mixed microbial communities. Cells or microorganisms not in a fixed relationship with a wall of a channel, such as a cell suspension, may be fed through channels of the apparatus, but it is preferred that at least one culture is adhered to all, substantially all, or a part of at least one cell culture channel.

The cell culture or cultures are preferably established prior to conducting any experiments, although cultures may also be established during the experiment, and may be seeded and cultured prior to attaching the channels to the membrane, if this construction method is used, or may be introduced through the entrance means and allowed to attach to the channel, varying nutrient flow as desired while establishing a culture.

In one aspect, there is provided a modular apparatus, preferably based on microfluidic principles, that allows the partitioned cultivation of cells and cell cultures, such as human cell lines and microbial communities, including sampled human microbial communities, while simultaneously permitting molecular interactions between adjacent cultures via a permeable or semipermeable membrane. Supported membranes as described above may be used in this aspect.

It will be appreciated that the molecular interactions permitted by the apparatus of the invention can be probed and analysed in any manner desired, such as by high-resolution molecular methods, including genomics, transcriptomics, proteomics, metabolomics, or other molecular analysis techniques, or other imaging or spectroscopy techniques. In particular, the apparatus allows separation of the individual channels following, for example, an experiment, thereby allowing subsequent biomolecular extractions from the respective cell contingents. It will be appreciated that separation may be effected by cutting the layers apart, or by constructing the apparatus in such a way as to permit disassembly after use. This may be achieved by heating or sonicating the apparatus after use, where such was used to achieve initial bonding, and where it will not significantly adversely affect the results of the experiment, or may be achieved by using an adhesive that does not fully set, or simply by unclamping the apparatus, if a clamp is used, for example. Other means for taking the apparatus apart will be apparent to those skilled in the art.

In one embodiment, the whole or part of the apparatus may be immersed in liquid nitrogen following an experiment. The frozen constituents may then be subjected to channel separation and biomolecular extractions on the cell populations present in the respective channels, for example.

It is an advantage of the present invention that the co-culture of different cellular contingents such as human-derived cells, including microorganisms, is now possible in close spatial and chemical proximity, and that it can allow, for example, the systematic interrogation of human and microbial molecular interactions to assess their potential for determining human health and disease states.

Particular advantages of the present invention are:

1) By providing separate culture channels, the apparatus of the invention is not limited to the observation of the effect of probiotic strains on human cells, such as gut epithelial cells, and not only non-pathogenic strains but pathogenic microorganisms may be cultured in channels adjacent human cell culture channels;
2) It is possible to co-culture anaerobic microorganisms in channels adjacent to those carrying human cells. It is of particular interest to observe the interaction of anaerobes with human cells, as these represent over 60% of the gut microbiota;
3) It is possible to carry out targeted perturbations on the separated individual cell cultures;
4) It is possible to carry out separate biomolecular extractions on each of the separate cell cultures for the first time;
5) It is possible to duplicate gut in vivo physiology and selectivity via e.g. mucin composition;
6) It is not necessary to prevent bacterial overgrowth by flow-based flushing of unbound bacteria. This can be prevented by separating the cultures using membranes;
7) It is possible to co-culture multiple cell types in parallel channels. Nutrient media may be flowed in dedicated channels or through the cell culture channel, as desired;
8) It is possible to incorporate sensors, such as oxygen sensors, thereby facilitating monitoring and controlled maintenance of the local micro-environment; and/or
9) It is possible to closely simulate in vivo conditions and, thus, to sustain microbial dysbiotic cultures and use these for diagnostic and tailored therapeutic purposes.

A preferred embodiment is adapted to allow the partitioned cultivation of human cell lines and sampled human microbial communities, while at the same time allowing molecular interactions between both contingents across a permeable membrane. The apparatus may be adapted to allow the design of in vitro models for several applications, such as in the human proximal colon, the human gastrointestinal tract, and human gastrointestinal tissue, and other physiological systems.

The apparatus of the present invention may be used to perform the co-culture of patient-derived human cells and coexisting microbial communities. It is within the scope of the present invention to establish representative human cell co-cultures, e.g. epithelial and neuronal cell lines in adjacent channels.

Further advantages of the present invention include one or more of the following: (i) improved surface adherence; (ii) more effective media supply, optionally in separate adjacent channels; (iii) juxtaposing of separate cell lines within diffusion distance (e.g. 6 μm) for facilitating cellular interactions and collection of metabolites and other by-products that can be analysed as desired.

Where there are multiple apparatus of the invention in sequence, as shown in FIG. 7, the pH of the medium may be adjusted before being fed into the next apparatus unit, for example, as it flows out of the small intestine microchannel (pH adjustment to 5.5) with the pH being allowed to evolve freely in the following channels. The pH may be adjusted using a CO₂/pH gas controller apparatus (Harvard Apparatus S.á.r.l, Les Ulis, France; FIG. 7B). The pH may also be recorded following the ascending and transcending colon microchannels, for example, and it is also possible to incorporate pH adjustment channels for the effluent from other channels.

Apart from ports for the introduction of medium into the apparatus, additional ports for specific experiments can also be included in the design. The dimensions of the microchannels can preferably be chosen to take advantage of the full surface area of the circular membrane and to provide ample surface area (approximately 840 mm² per microchannel) for the culture of appropriate cell numbers. Obtaining representative biomolecular fractions for downstream high-throughput omics typically requires 10⁶ human cells, which translates to a microchannel surface area of around 2400 mm², which in turn may require the stacking of up to three microchannel apparatus on top of each other (FIG. 7A).

FIG. 7 illustrates apparatus of the invention. (A) Human proximal colon model allowing the partitioned cultivation of human and microbial cell populations with molecular interactions possible through a permeable membrane. The apparatus design is modular to facilitate appropriate cell culture volumes to be obtained. (B) Human gastrointestinal tract model highlighting the modular nature and multiplexing ability of the apparatus. Approximate medium residence times are indicated for each compartment. (C) Human gastrointestinal tissue model showing the co-culture of several human cell lines types, e.g. epithelial cells and neurons, in conjunction with mixed microbial communities.

In one embodiment, prior to inoculation, the side of the semipermeable membrane exposed to microbial consortia may be layered with mucus, for example, obtained from the HT29-MTX human cell line [Lesuffleur et al., 1990; Coconnier et al., 1992]; resected human intestinal tissue [Vesterlund et al., 2006]; or with porcine mucin gel [Macfarlane et al., 2005], to assist initial microbial adhesion (FIG. 7A). Mucus (mucin) may be further supplied to the microbial community throughout the period of incubation by inclusion in the growth medium or by secretion by HT29-MTX cells in the human cell channel and subsequent diffusion into the microbial cell channel. The pore size of mucus is typically large enough for it not to prevent diffusion of biomolecules [Shen et al., 2006]. Consequently, efficient molecular exchange can be maintained across the whole membrane-mucus layer.

Fluidic movement can be activated, for example, by using an external syringe pump for precise liquid delivery which in turn can be controlled using a digital controller programmed with suitable software, such as the LabView software package (National Instruments, Austin, Tex., USA). The pumps preferably interface with the apparatus using a polyether ether ketone (PEEK)/silicone tubing connection to provide a tight and reliable seal [Estes et al., 2009], although the skilled person will be able to provide any suitable pump and connector. The apparatus and pump can be placed in an incubator and controlled by an external computer running an automated LabView script to direct media exchange [Hopwood et al., 2010]. For a human proximal colon apparatus, a flow rate of 7.3 μl/h can be used to guarantee a medium exchange rate of 52 h. For other apparatus, flow rates can be adjusted according to apparatus designs and/or layouts. However, in all cases, it is generally preferred to maintain the flow rate sufficiently low to avoid excessive detachment of cells due to shear stress. Before any culture experiments are carried out, it is generally desirable to perform partition tests by introducing molecules and particles of specific sizes into the medium and measuring if they are transferred across the membrane.

In a preferred embodiment, representative human cell lines that are well established cellular models and that, in the human body would naturally be in contact with mixed microbial communities, are selected for inoculation of the apparatus' human cell compartment(s). Faecal inoculate can be obtained from human volunteers, preferably in a healthy or defined diseased state. Following successful co-culture of the human cell lines in conjunction with the mixed microbial communities, cultivation involving sampled human cells/tissue and associated mixed microbial communities may also be undertaken, such as to emulate healthy or diseased states. Such samples can be obtained either by direct sampling or during routine medical procedures, e.g. gastroscopy or colonoscopy.

In this embodiment, specialised media are preferably used for the culturing of both cell populations. Initially, it is preferable that only human epithelial cells (9:1 mixture of Caco-2 [Hidalgo et al., 1989] and HT29-MTX [Lesuffleur et al., 1990] cells) are grown in the apparatus until a fully differentiated cell monolayer is formed. Cell lines can be obtained from the American Type Culture Collection (ATCC; Manassas, Va., USA). For human cell culture, Dulbecco's modified Eagle's medium (DMEM) can be flowed through both compartments. Following the establishment of stable cell monolayers (as determined by optical microscopy; expected after approximately 2-3 weeks), a complex medium that represents terminal ileal chyme [Gibson et al., 1988; van Nuenen et al., 2003] can be flowed through the microbial channel. Following equilibration, the microbial cell culture channels can be seeded with fresh faecal inoculate [Macfarlane et al., 2005]. Following the establishment of microbial communities (as determined by optical microscopy, for example), the human cell culture medium can be modified to just include inorganic salts as buffering agents. The apparatus can then be operated until the establishment of a stable functional state. The established microbial communities can be monitored by a combination of microscopy, high-resolution molecular microbial community profiling, and metabolomics to provide a base line for the following apparatus setups and experimental conditioning.

Oxygen concentrations may be measured and modelled by microfluidic diffusion analysis [Skolimowski et al., 2010]. The DMEM and buffer solution can subsequently be adjusted by using a defined length of slightly gas permeable silicone tubing through which the solutions can be flowed prior to introduction into the human cell channel. For example, it may be desirable to make the human cell culture channel from oxygen permeable polydimethylsiloxane (PDMS) instead of polycarbonate. Conversely, nitrogen gas can be bubbled through the microbial growth medium prior to introduction into the syringe and gas impermeable PEEK tubing can be used to establish complete anaerobic conditions.

Thus, the present invention further provides a method for modelling the interaction between two or more cell cultures, comprising establishing said cultures separately in cell cultivation channels of apparatus as defined herein.

In one embodiment nutrient media for at least one cell culture is supplied via a perfusion channel provided adjacent the cell cultivation channel and separated therefrom by a permeable or semipermeable membrane. Separately, or in addition thereto, nutrient media for at least one cell culture is supplied via the entrance and exit means of the cell cultivation channel.

In a preferred embodiment, one cell culture is a mammalian, preferably human, tissue, such as Caco-2, especially with HT29-MTX, and the other cell culture is a microbial colony, such as a consortium, especially a biofilm.

In another embodiment, the cell cultures include first and second cell cultures, and a first cell culture is pathogenic to a second cell culture.

In a preferred method, a first cell culture is aerobic and a second cell culture is anaerobic.

In the methods of the invention, it is preferred to monitor interactions between said cell cultures by monitoring means, such as are described hereinabove.

In the methods of the invention, it is preferred to monitor oxygen levels in at least one cell culture or perfusion channel by oxygen level monitoring means.

In another preferred embodiment, a plurality of apparatus units as defined herein is fluidically connected in series, optionally with each said apparatus having the same or different cell cultures and/or nutrient media supplies.

The apparatus of the present invention may be used in a great many applications, of which a few examples are as follows:

1. Development of individual- and enterotype-specific gastrointestinal models;
2. Elucidation of microbial association with different mucin types;
3. Study of gut microbiome modulation, e.g. through a faecal transplantation process;
4. Elucidation of the impact of microbiome modulating pre- and pro-biotics;
5. Elucidation of pathogenesis by viral and microbial co-infections;
6. Diagnosis of viral and microbial co-infections through culturing of patient-derived samples;
7. Investigation of the effect of the microbiome on drug bioavailability, drug intake, and the catabolism of chemicals or drugs;
8. Tailoring of drug therapy through culturing of patient-derived samples;
9. Investigation of impact of long- and short-term dietary habits;
10. Investigation of impact of long- and short-term antibiotic therapy;
11. Tailoring of antibiotic therapy for the treatment of infectious diseases;
12. Investigation of impact of radiation dose on gut microbiota and human cells;
13. Tailoring of radiation dose for radiation therapy through culturing of patient-derived samples;
14. Investigation of impact of targeted perturbations of the healthy or diseased microbiome with specific small molecules, peptides, proteins and nucleic acids;
15. Investigation of impact of microbial dysbiosis on metabolic disorders, such as obesity, or diabetes;
16. Diagnosis of microbial dysbiosis-mediated metabolic disorders through culturing of patient-derived samples;
17. Investigation of the role of dysbiosis in cancer, for example, pancreatic cancer and gynecological cancers;
18. Diagnosis of microbial dysbiosis-mediated cancers;
19. Investigation of the impact of microbial dysbiosis on any disease linked to microbial dysbiosis; and
20. Diagnosis and personalised treatment of any microbial dysbiosis-mediated disease.

### BRIEF DESCRIPTION OF THE DRAWINGS

The invention is illustrated by the accompanying drawings, in which:

FIG. 1 is a perspective view of a culture apparatus in accordance with the present invention;

FIG. 2 is a schematic top view of culture apparatus of the invention;

FIG. 3 is a schematic aerial view and cross section of culture apparatus of the invention;

FIG. 4A is a schematic cross section of a culture apparatus according to one embodiment (Design 1);

FIG. 4B is a schematic cross section of a culture apparatus according to another embodiment (Design 2);

FIG. 4C is a schematic cross section of a culture apparatus according to another embodiment showing the microbial co-culture apparatus with dedicated perfusion channel (Design 3);

FIG. 5 a schematic cross section of a culture apparatus according to another embodiment (Design 4);

FIG. 6 shows a human in vitro proximal colon model;

FIG. 7A illustrates a human proximal colon model allowing the partitioned cultivation of human and microbial cell populations with molecular interactions possible through the semipermeable membrane;

FIG. 7B illustrates a human gastrointestinal tract model highlighting the modular nature and multiplexing ability of the apparatus. Approximate medium residence times are indicated for each compartment;

FIG. 7C illustrates a human gastrointestinal tissue model showing the co-culture of several human cell lines types, e.g. epithelial cells and neurons, in conjunction with mixed microbial communities; and

FIG. 8 illustrates an apparatus and method according to the present invention.

### DETAILED DESCRIPTION OF THE INVENTION

The following represent preferred embodiments of the invention, but are not limiting thereon.

### i) Design and Fabrication of Human In Vitro Proximal Colon Model.

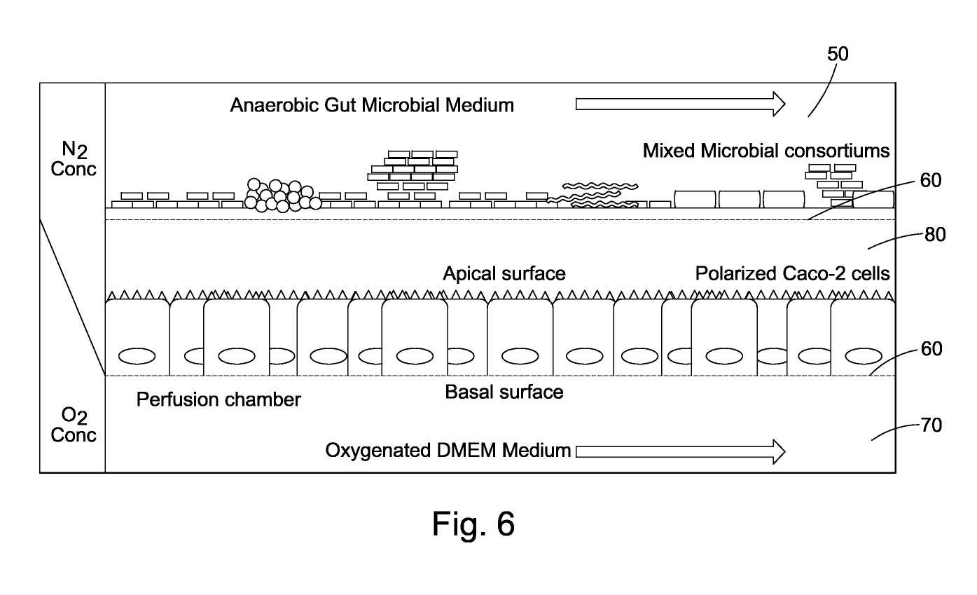

In order to determine the degree of molecular interaction between human and microbial cell populations, it is necessary to have a means of co-culturing both cell types in close proximity to each other without them being in actual direct physical contact with one another. Considerations of cost, reliability, throughput, multiplexing ability and flexibility of fabrication clearly favour a microfluidic architecture [Whitesides, 2006]. For initial prototyping and testing of the proposed microfluidics-based co-culture apparatus architecture, a three-compartment apparatus was assembled which allowed the modelling of the human proximal colon, i.e. combined ascending and transverse colon (FIG. 6). FIG. 6, (50) shows the microbial channel, (80) shows the human channel, (60) shows the membrane, (10) is the apparatus, (20) the entrance and (40) the exit means, and (70) is the perfusion channel, or may be used for another culture channel.

The circular apparatus was designed using the AutoCAD software package (Autodesk, San Rafael, Calif., USA). The apparatus was created by bonding together separate spiral microchannels made of polycarbonate polymer. These channels were formed by computer numerically controlled (CNC) machining of 0.2 mm and 0.5 mm thick polycarbonate plate stock [Becker and Gärtner, 2000]. Other designs used 1 mm thick stock, with channels 0.2 or 1 mm deep and 0.8 mm wall thickness, while other designs used 0.25 or 0.5 mm thick stock with channels 0.2 or 0.25 mm deep and 0.3 mm wall thickness. The use of polycarbonate allows for accurate control of the respective levels of dissolved oxygen within both channels, i.e. aerobic conditions in the human cell culture channel and anaerobic conditions in the microbial cell culture channel.

### Design 1

The channels have a wall thickness of 800 μm to maximise structural integrity. The size of each microchannel is 380 μl, formed by 200 μm deep, 4 mm wide and 0.5 m long channels fit into a circular area of a diameter of 70 mm. The channels are partitioned by permeable polycarbonate membranes (70 mm in diameter, nanoporous with a thickness of 6 μm; Advantec MFS Inc., Dublin, Calif., USA). The microchannels are bound to either side of the permeable membrane using fitted and biologically compatible double-sided pressure sensitive adhesive (Adhesives Research, Glen Rock, Pa., USA).

### Design 2

The channels have a wall thickness of 800 μm to maximise structural integrity. The size of each microchannel is 170 μl, formed by 200 μm deep, 4 mm wide and 0.2 m long channels fit into a circular area of a diameter of 46 mm. The channels are partitioned by semipermeable polycarbonate membranes (46 mm in diameter, nanoporous with a thickness of 6 μm; Advantec MFS Inc., Dublin, Calif., USA). The microchannels are again bound to either side of the semipermeable membrane using fitted and biologically compatible double-sided pressure sensitive adhesive. The apparatus design was modified to account for modifications in the downstream biomolecular extraction protocol and its requirements in terms of maximum loading capacity of the chromatographic columns used for extractions of DNA, RNA and proteins.

### Design 3

A dedicated perfusion channel, separated by means of a semipermeable membrane, is introduced under the cell culture channel, e.g. in which Caco-2 cells are cultured, which provides diffusion-dominant perfusion to the Caco-2 cells, thereby mimicking the in vivo perfusion dynamics, and allowing perfusion of the basolateral surface of the Caco-2 cells. There are significant advantages with this kind of perfusion mechanism [Shah et al., 2011]. First, intestinal epithelial cells are normally perfused via diffusion in vivo, so that this mode of perfusion helps to recreate the extracellular matrix conditions for the cells. Secondly, it has already been shown using transwell membrane inserts that diffusion based perfusion to basolateral surface speeds up epithelial cell growth, differentiation, and polarisation, thereby reducing cell culture time from 21 days to 7 days [Yamashita et al., 2002], which is significant improvement on assay time, and reduces other costs associated with reagents, for example. Finally, as the cells are perfused using dedicated perfusion channels, they are prevented from experiencing shear stress that may occur in Designs 1 and 2 without a separate perfusion channel. This can be advantageous for cell types in which shear stress can change the gene expression profile of cells. In such cases, the membrane that borders the perfusion channel preferably has a mean pore size of between 0.5-2 μm. In general, membranes separating cell cultures, especially separate cultures of human and microbial cells, preferably have pore sizes in the nanometre range, such between 1 and 20 nm, preferably between 1 and 10 nm.

In general, it will be appreciated that dedicated perfusion channels do not need to have a cross section of 1 mm² or less, as microfluidics is of less concern for such channels.

### Design 4

In this design, the individual channels are separated using a semi-permeable membrane. The apparatus design has been modified to facilitate easy optical analysis of the co-cultures. The outermost polycarbonate layers are reduced to 0.2 mm thickness, while the middle layers are reduced to 0.5 mm thickness. For co-culture experiments, bubble traps for easy removal of the bubbles escaping from oxygenated DMEM medium [Zheng et al., 2010] can be used, thereby overcoming a common problem in microfluidic devices. The channel walls covering the microfluidic channel have 2 mm holes which are sealed by a cover glass incorporating optical sensing element (optodes) for sensing oxygen concentration in the medium in different channels [Kuhl et al., 2008]. The polycarbonate layers in this and in other aspects and embodiments may be designed with one or more glass viewing windows to facilitate easy optical inspection of the co-cultures.

In each of the above designs, the use of the membrane can prevent typical problems encountered in co-cultures, e.g. microorganisms rapidly taking over human cells due to pronounced differences in growth rates, and, thus, can allow prolonged and sustained culture of human and microbial cells. In addition, the apparatus of the invention allows efficient perfusion of media in addition to allowing molecular probing of both cell contingents.

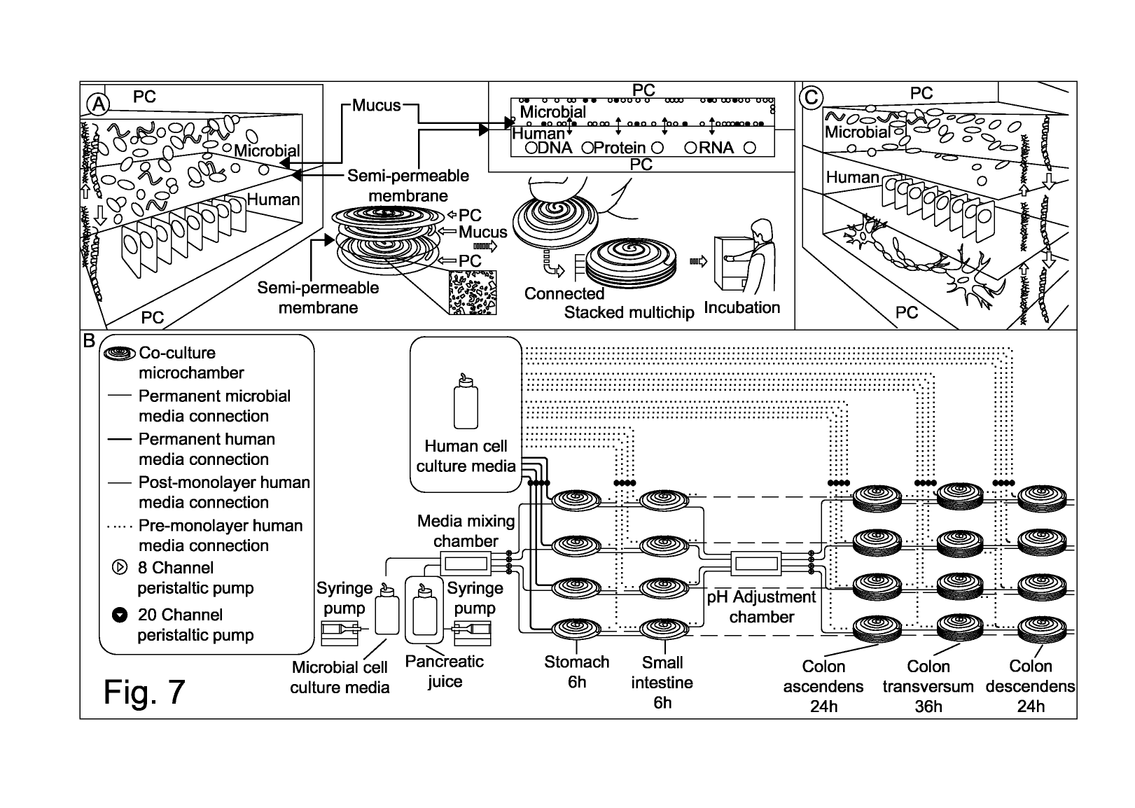

The preferred human proximal colon apparatus model may be expanded with additional apparatus arranged in series to simulate the human gut (FIG. 7B) as well as the stacking of several human cell channels to model human gastrointestinal tissue (FIG. 7C).

### Use of Design 3

The apparatus of Design 3 was used to test the co-culture of Caco-2 and bacterial cells. Apart from the individual cell contingents, the additional channel underneath the Caco-2 cells was used to perfuse the basal surface of the Caco-2 cells via diffusion through the membrane. After the Caco-2 cells were initially cultured for 7 days with medium containing Penicillin-Streptomycin, the cells were cultured for 24 h with medium excluding antibiotics prior to co-culture. The bacterial cells (E. coli strain Dh5a and faecal microbial consortium) were inoculated on top of a porcine mucin layer in the bacterial culture channel and perfusion was stopped to both the cell types. After 3 h, the non-adhered bacteria were washed off with PBS and the apparatus was analysed with optical microscopy after 2 h.

For inoculation of the human cell microchannels, representative human cell lines that form monolayers may be chosen, e.g. the AGS [Barranco et al., 1983], Kato III [Sekiguchi et al., 1978] or MKN28 [Romano et al., 1988] cell lines, for the stomach compartment, and the Caco-2 and HT29-MTX cell line mixtures for the subsequent compartments. Animal, mammal, or human cells derived from patient samples may also be used as inoculum. In the human intestinal model, stable monolayers of cells can be allowed to form in the microchannels before microbial cell culture medium comprising SHIME feed and artificial pancreatic juice [Van den Abbeele et al., 2010] fed through the successively arranged microbial community channels. In order to provide a supply of sufficiently rich medium to the human cell lines, each microchannel may be supplied with fresh DMEM. Following equilibration of the system, fresh human faecal samples can be used as inoculate and the human cell culture medium rarefied. The rarefied medium can be fed through the whole system following valve adjustment (FIG. 7B) and only discarded after the cascade of apparatus. Following the establishment of a stable functional state within the respective mixed microbial communities [Van den Abbeele et al., 2010], specific measurements can be carried out on the regions of interest.

FIG. 7C illustrates amodular microfluidics-based apparatus design that recreates a multi-layered human gastrointestinal tissue model, and provides a tissue model of the human stomach and of the human proximal colon to allow the investigation of effects of molecular cross-talk on e.g. neural cells. For both gastrointestinal compartments, a human and microbial cell co-culture apparatus as described above is assembled and which is representative of the human proximal colon and of the human stomach, with the addition of an additional microchannel layer that allow the cultivation of human neuronal cell lines or others, e.g. immune cells. For the human stomach tissue model, the volume of the apparatus compared to the human gastrointestinal tract model may be increased by including three additional microchannel stacks to provide sufficient cell numbers for downstream omic analyses. The overall setup and culture conditions are analogous to the gastrointestinal tract model, except for the lack of separate large intestinal compartments. Human neuronal cell lines, e.g. the Lund human mesencephalic (LUHMES) cell line [Lotharius et al., 2002], can be grown in tandem with the human epithelial cell lines in standard DMEM. Following the establishment of stable cell populations in both the epithelial and neuronal cell culture microchannels, the microbial culture medium can be introduced followed by inoculation. At this point, the DMEM can again be rarefied.

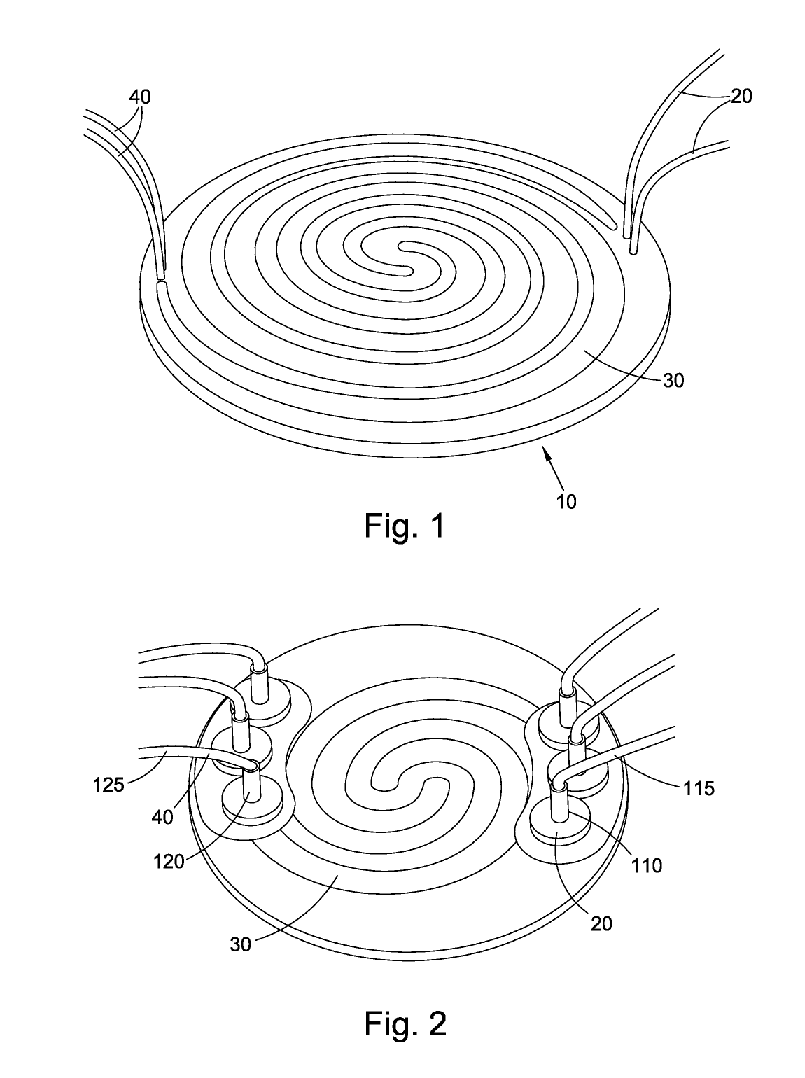

FIG. 1 shows a cell culture apparatus (10) comprising two adjacent cell cultivation channels (30) separated by a permeable or semi-permeable membrane (not shown). Entrance means (20) and exit means (40) provide fluidic access to each channel. It will be appreciated that, if one channel is contra-flow, then one of the two entrance means (20) will become an exit means (40) and the corresponding exit means (40) will become entrance means (20). Nutrient or assay media may be introduced via entrance means (20) and removed via exit means (40).

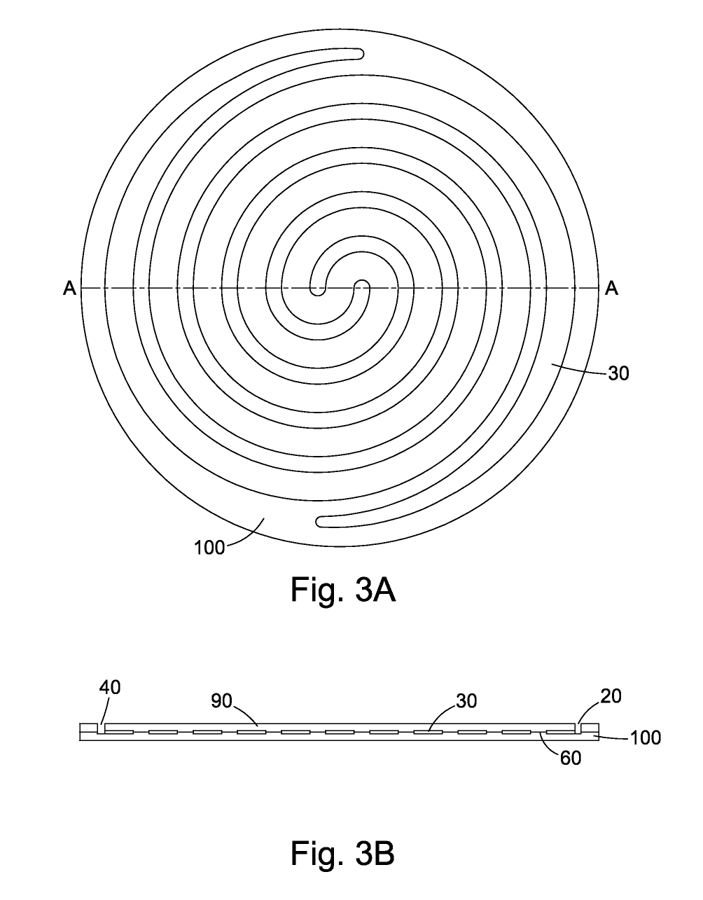

FIG. 2 shows an elevated view of the apparatus of the invention in use, wherein the reference numerals have the same meaning as for FIG. 1. It can be seen that entrance means (20) each comprises a nipple (110) onto which tubing (115) can be secured by a push fit. Likewise, exit means (40) comprises nipple (120) over which tubing (125) can be secured by a push fit. It will be appreciated that each of the channels making up the channel bundle (30) may have one, or more than one, entrance means (20) and exit means (40), and that the number of entrance means 20 does not need to match the number of exit means (40).

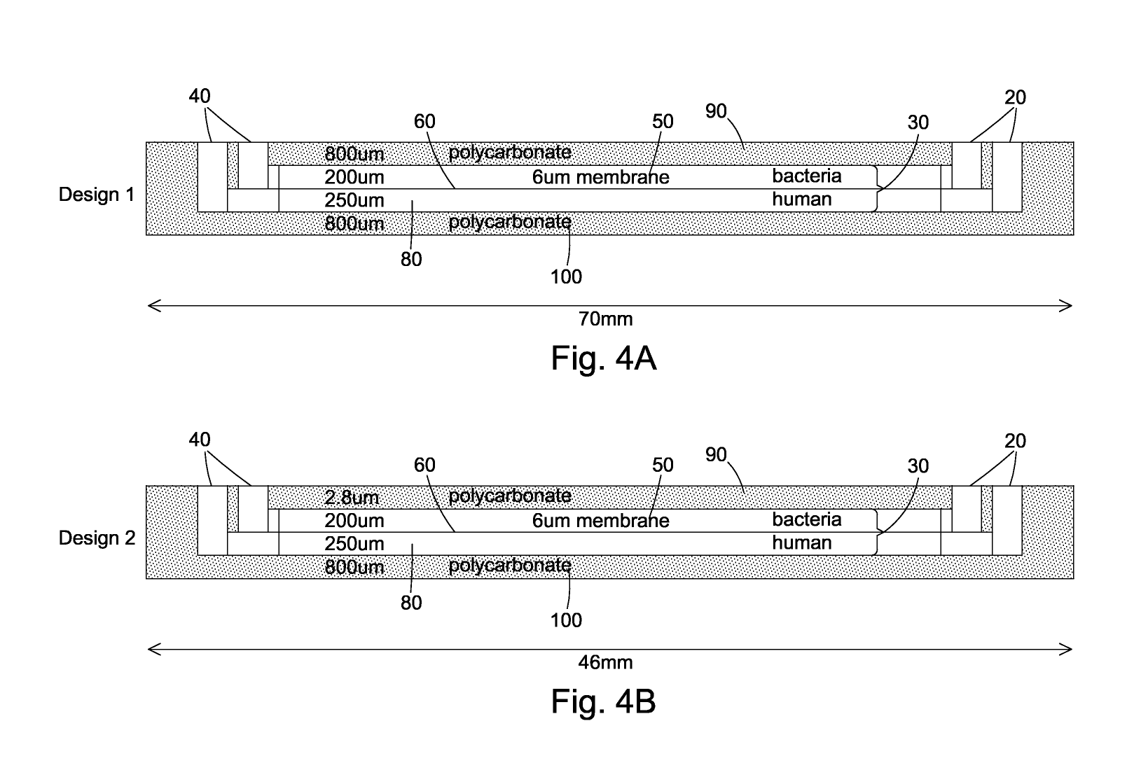

FIG. 3A depicts a plan view from underneath of a clear, polycarbonate layer (100) containing channels (30).

FIG. 3B is a cross-section on A-A of FIG. 3A, and shows entrance (20), exit (40) and channels (30). Top layer (90) is shown in juxtaposition with bottom layer (100) and sandwiching membrane (60) which separates channels (30).

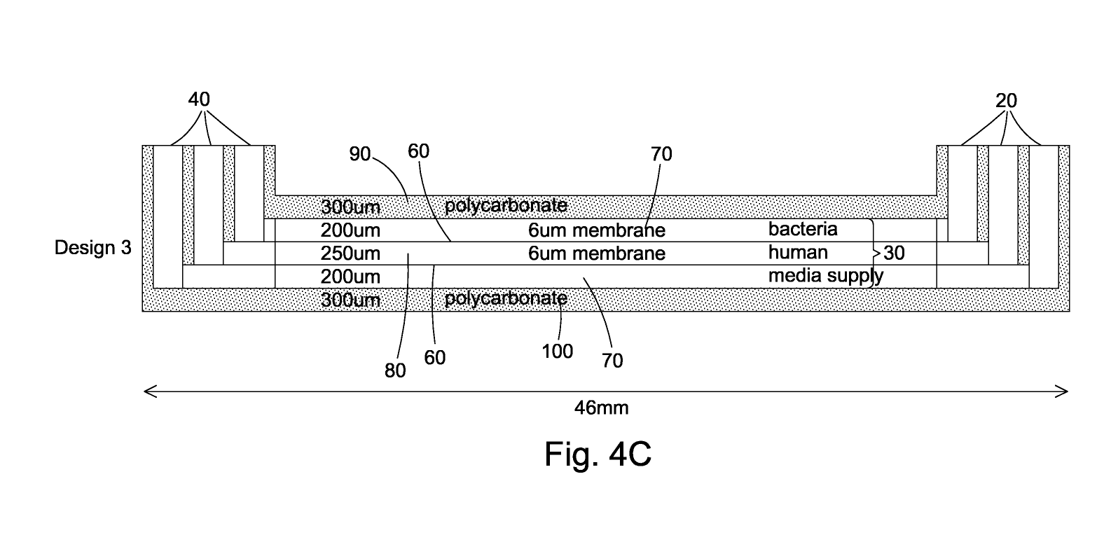

FIG. 4A illustrates Design 1, FIG. 4B illustrates Design 2, and FIG. 4C illustrates Design 3, said Designs being as described hereinabove. The numerals in FIGS. 4A, 4B, and 4C are as for FIGS. 1 to 3. A top, typically microbial, microchannel (5) is separated from a human cell culture channel (80) by semi-permeable membrane (60). In FIG. 4C, human microchannel (80) is separated from media supply, or perfusion, channel (70) by a permeable or semi-permeable membrane (60).

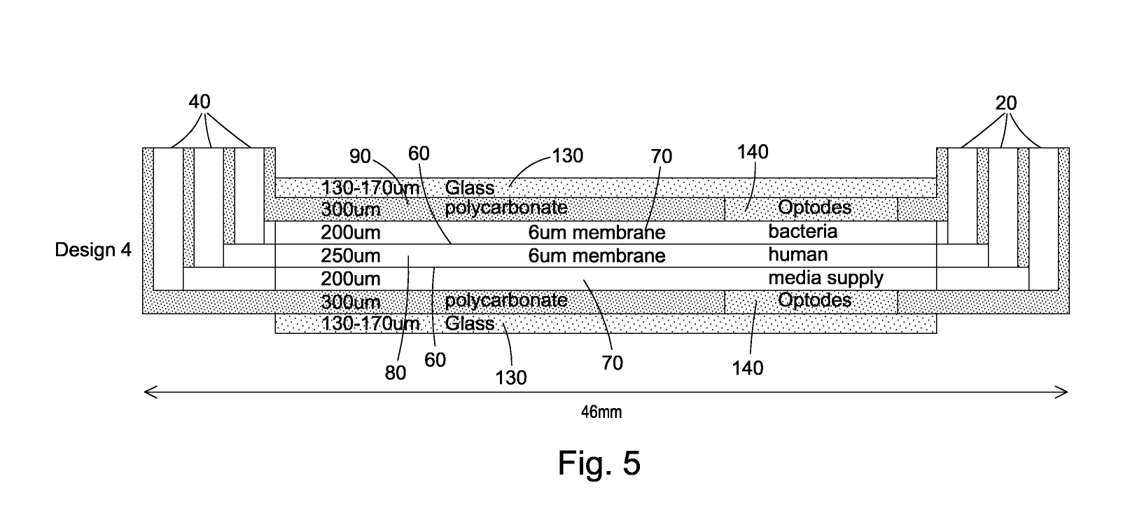

FIG. 5 illustrates Design 4, wherein numerals are as in previous Figures. In addition, optical sensors (optodes) are shown at (140), and the exposed surfaces of the apparatus are covered by glass cover slips (130).

FIG. 6 illustrates an embodiment associated with Design 3 and shows how a mixed consortium layer (50) can be co-cultivated with human Caco-2 cells in microchannel (80), separated by membrane (60). The effect of the consortia on the human cells can then be monitored by monitoring the chemical and any other measurable response of the human cells and vice versa. This may be by the presence of monitors, or by sampling the human or microbial cultures. In addition, any exhausted medium may also be monitored for relevant indicators.

FIG. 7 illustrates various modular embodiments of the invention. In FIG. 7A, there is illustrated apparatus pre-assembly, showing the constituent layers, and also showing assembly of apparatus units in multiples. Such assembly may either be in series, wherein selected media flow from one unit to the next, or may be in parallel, wherein each unit has its own media supply. Where there is more than one media supply, it is also possible to use mixed series and parallel supplies, wherein one supply may be fed from one unit to the next, whilst another supply, such as oxygenated medium, may be supplied in parallel.

FIG. 7B illustrates how units of apparatus of the invention may be used to model the human gut system. It can be seen that, in this system, four arrays of units are provided, each array being in series, and each series array being in parallel with the next array. A pH adjustment chamber is provided after the small intestine model.

FIG. 7C illustrates a cross-section of an apparatus, such as is illustrated in Design 3, and shows three culture microchannels, one microbial, one human epithelium channel and one nervous tissue channel.

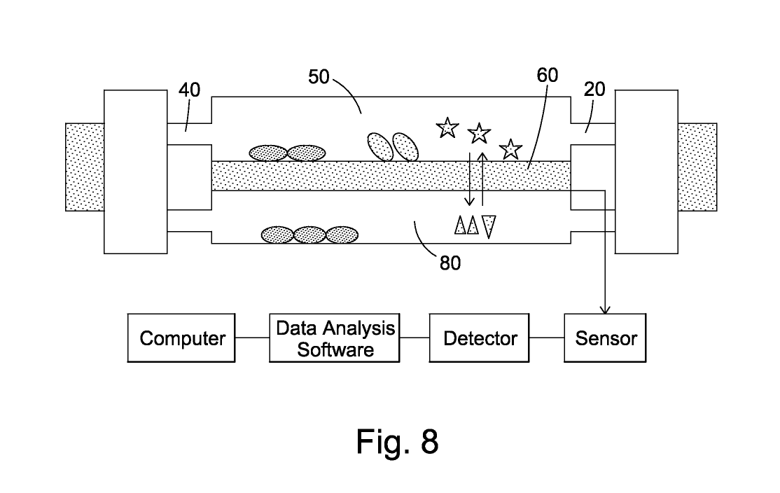

FIG. 8 generally illustrates a simple embodiment of the present invention, wherein microbial consortia present at (50) are able to interact with human cells present at (80) via semipermeable membrane (60) a sensor/detector/data analysis software/computer array is located as indicated to monitor the interaction between the microbes at (50) and the human cells at (80).

### REFERENCE LIST

Andersson A F, Lindberg M, Jakobsson H, Bäckhed F, Nyrén Pl, Engstrand L (2008) Comparative analysis of human gut microbiota by barcoded pyrosequencing. PLoS ONE3: e2836.
Artis D (2008) Epithelial-cell recognition of commensal bacteria and maintenance of immune homeostasis in the gut. Nature Reviews Immunology 8: 411-420.
Barranco S C, Townsend C M, Casartelli C, Macik B G, Burger N L, Boerwinkle W R, Gourley W K (1983) Establishment and characterization of an in vitro model system for human adenocarcinoma of the stomach. Cancer Research 43: 1703-1709.
Becker H, Gärtner C (2000) Polymer microfabrication methods for microfluidic analytical applications. Electrophoresis 21: 12-26.
Bhatia S N, Balis U J, Yarmush M L, Toner M (1999) Effect of cell-cell interactions in preservation of cellular phenotype: cocultivation of hepatocytes and nonparenchymal cells. The FASEB Journal 13: 1883-1900.
Biehler E, Bohn T (2010) Methods for assessing aspects of carotenoid bioavailability. Current Nutrition and Food Science 6: 44-69.
Braak H, de Vos R A I, Bohl J, Del Tredici K (2006) Gastric a-synuclein immunoreactive inclusions in Meissner's and Auerbach's plexuses in cases staged for Parkinson's disease-related brain pathology. Neuroscience Letters 369: 67-72.
Candela M, Guidotti M, Fabbri A, Brigidi P, Franceschi C, Fiorentini C (2010) Human intestinal microbiota: cross-talk with the host and its potential role in colorectal cancer. Critical Reviews in Microbiology 37: 1-14.
Chervonsky A V (2010) Influence of microbial environment on autoimmunity. Nature Immunology 11: 28-35.
Coconnier M H, Klaenhammer T R, Kerneis S, Bernet M F, Servin A L (1992) Protein-mediated adhesion of Lactobacillus acidophilus BG2FO4 on human enterocyte and mucus-secreting cell lines in culture. Applied and Environmental Microbiology 58: 2034-2039.
Coenye T, Nelis H J (2010) In vitro and in vivo model systems to study microbial biofilm formation. Journal of Microbiological Methods 83: 89-105.
Déat E, Blanquet-Diot S, Jarrige J-F, Denis S, Beyssac E, Alric M (2009) Combining the dynamic TNO-gastrointestinal tract system with a Caco-2 Cell Culture Model: application to the assessment of lycopene and !-tocopherol bioavailability from a whole food. Journal of Agricultural and Food Chemistry 57: 11314-11320.
Denef V J, Mueller R S, Bonfield J F (2010) AMD biofilms: using model communities to study microbial evolution and ecological complexity in nature. ISME J4: 599-610.
Dicksved J, Halfvarson J, Rosenquist M, Jarnerot G, Tysk C, Apajalahti J, Engstrand L, Jansson J K (2008) Molecular analysis of the gut microbiota of identical twins with Crohn's disease. The ISME Journal 2: 716-727.
Donoghue H D (1990) Can the colonisation resistance of the oral microflora be enhanced? Microbial Ecology in Health and Disease 3: i-iv.
Estes M D, Ouyang B, Ho S, Ahn C H (2009) Isolation of prostate cancer cell subpopulations of functional interest by use of an on-chip magnetic bead-based cell separator. Journal of Micromechanics and Microengineering 19: 095015.
Frank D N, St. Amand A L, Feldman R A, Boedeker E C, Harpaz N, Pace N R (2007) Molecular-phylogenetic characterization of microbial community imbalances in human inflammatory bowel diseases. Proceedings of the National Academy of Sciences 104: 13780-13785.
Frimat J P, Becker M, Chiang Y, Marggraf U, Janasek D, Hengstler J, Franzke J, West J (2011) A microfluidic array with cellular valving for single cell co-culture. Lab Chip 11: 231-7
Gibson G R, Cummings J H, Macfarlane G T (1988) Use of a three-stage continuous culture system to study the effect of mucin on dissimilatory sulfate reduction and methanogenesis by mixed populations of human gut bacteria. Applied and Environmental Microbiology 54: 2750-2755.
Gill S R, Pop M, DeBoy R T, Eckburg P B, Turnbaugh P J, Samuel B S, Gordon J I, Relman D A, Fraser-Liggett C M, Nelson K E (2006) Metagenomic analysis of the human distal gut microbiome. Science 312: 1355-1359.
Giongo A, Gano K A, Crabb D B, Mukherjee N, Novelo L L, Casella G, Drew J C, Ilonen J, Knip M, Hyoty H, Veijola R, Simell T, Simell O, Neu J, Wasserfall C H, Schatz D, Atkinson M A, Triplett E W (2011) Toward defining the autoimmune microbiome for type 1 diabetes. The ISME Journal 5: 82-91.
Goñi I, Serrano J, Saura-Calixto F (2006) Bioaccessibility of β-Carotene, Lutein, and Lycopene from Fruits and Vegetables. Journal of Agricultural and Food Chemistry 54: 5382-5387.
Goodacre R (2007) Metabolomics of a superorganism. The Journal of Nutrition 137: 259S-266S.
Gosalbes M J, Durbán A, Pignatelli M, Abellan J J, Jiménez-Hernández N, Pérez-Cobas A E, Latorre A, Moya A (2011) Metatranscriptomic approach to analyze the functional human gut microbiota. PLoS ONE 6: e17447.
Hehemann J-H, Correc G, Barbeyron T, Helbert W, Czjzek M, Michel G (2010) Transfer of carbohydrate-active enzymes from marine bacteria to Japanese gut microbiota. Nature 464: 908-912.
Hidalgo, I J, Raub, T J, Borchardt, R T (1989) Characterization of the human colon carcinoma cell line (Caco-2) as a model system for intestinal epithelial permeability. Wiley, Hoboken, N.J., USA.
Hooper L V, Midtvedt T, Gordon J I (2002) How host-microbial interactions shape the nutrient environment of the mammalian intestine. Annual Review of Nutrition 22: 283.
Hopwood A J, Hurth C, Yang J, Cai Z, Moran N, Lee-Edghill J G, Nordquist A, Lenigk R, Estes M D, Haley J P, McAlister C R, Chen X, Brooks C, Smith S, Elliott K, Koumi P, Zenhausern F, Tully G (2010) Integrated microfluidic system for rapid forensic DNA analysis: sample collection to DNA profile. Analytical Chemistry 82: 6991-6999.
Husebye E, Hellstrom P, Midtvedt T (1994) Intestinal microflora stimulates myoelectric activity of rat small intestine by promoting cyclic initiation and aboral propagation of migrating myoelectric complex. Digestive Diseases and Sciences 39: 946-956.
Jansson J, Willing B, Lucio M, Fekete A, Dicksved J, Halfvarson J, Tysk C, Schmitt-Kopplin P (2009) Metabolomics reveals metabolic biomarkers of Crohn's disease. PLoS ONE 4: e6386.
Kim J, Hegde M, Jayaraman A (2010) Co-culture of epithelial cells and bacteria for investigating host-pathogen interactions. Lab on a Chip 10: 43-50.
King C, Sarvetnick N (2011) The incidence of type-1 diabetes in NOD mice is modulated by restricted flora not germ-free conditions. PLoS ONE 6: e17049.
Kühl M, Polerecky L (2008) Functional and structural imaging of phototrophic microbial communities and symbioses. Aquatic Microbial Ecology 53: 99-118
Lebouvier T, Neunlist M, Bruley des Varannes S, Coron E, Drouard A, N′Guyen J-M, Chaumette T, Tasselli M, Paillusson S B, Flamand M, Galmiche J-P, Damier P, Derkinderen P (2010) Colonic biopsies to assess the neuropathology of Parkinson's disease and its relationship with symptoms. PLoS ONES: e12728.
Lesuffleur T C, Barbat A, Dussaulx E, Zweibaum A (1990) Growth adaptation to methotrexate of HT-29 human colon carcinoma cells is associated with their ability to differentiate into columnar absorptive and mucus-secreting cells. Cancer Research 50: 6334-6343.
Lindén S K, Driessen K M, McGuckin M A (2007) Improved in vitro model systems for gastrointestinal infection by choice of cell line, pH, microaerobic conditions, and optimization of culture conditions. Helicobacter 12: 341-353.
Lotharius J, Barg S, Wiekop P, Lundberg C, Raymon H K, Brundin P (2002) Effect of mutant-synuclein on dopamine homeostasis in a new human mesencephalic cell line. Journal of Biological Chemistry 277: 38884-38894.
Ma H, Liu T, Qin J, Lin B (2010) Characterization of the interaction between fibroblasts and tumor cells on a microfluidic co-culture device. Electrophoresis 31: 1599-605
Macfarlane G T, Macfarlane S (2007) Models for intestinal fermentation: association between food components, delivery systems, bioavailability and functional interactions in the gut. Current Opinion in Biotechnology 18: 156-162.
Macfarlane S, Woodmansey E J, Macfarlane G T (2005) Colonization of mucin by human intestinal bacteria and establishment of biofilm communities in a two-stage continuous culture system. Applied and Environmental Microbiology 71: 7483-7492.
Macpherson A J, Harris N L (2004) Interactions between commensal intestinal bacteria and the immune system. Nature Reviews Immunology 4: 478-485.
Manichanh C, Rigottier-Gois L, Bonnaud E, Gloux K, Pelletier E, Frangeul L, Nalin R, Jarrin C, Chardon P, Marteau P, Roca J, Dore J (2006) Reduced diversity of faecal microbiota in Crohn's disease revealed by a metagenomic approach. Gut55: 205-211.
McBain A J, Allen I L, Sima S, Geoffrey M G (2009) In vitro biofilm models: an overview. Advances in Applied Microbiology 69: 99-132.
Minekus M, Smeets-Peeters M, Bernalier A, Marol-Bonnin S, Havenaar R, Marteau P, Alric M, Fonty G, Huis in′t Veld J H J (1999) A computer-controlled system to simulate conditions of the large intestine with peristaltic mixing, water absorption and absorption of fermentation products. Applied Microbiology and Biotechnology 53: 108-114.
Molly K, Woestyne M, Verstraete W (1993) Development of a 5-step multi-chamber reactor as a simulation of the human intestinal microbial ecosystem. Applied Microbiology and Biotechnology 39: 254-258.
Morowitz M J, Denef V J, Costello E K, Thomas B C, Poroyko V, Relman D A, Banfield J F (2011) Strain-resolved community genomic analysis of gut microbial colonization in a premature infant. Proceedings of the National Academy of Sciences 108: 1128-1133.
Ochoa-Repáraz J, Mielcarz D W, Begum-Haque S, Kasper L H (2011) Gut, bugs, and brain: role of commensal bacteria in the control of central nervous system disease. Annals of Neurology 69: 240-247.
Park J, Kerner A, Burns M A, Lin X N (2011) Microdroplet-enabled highly parallel co-cultivation of microbial communities. PLoS ONE 6: e17019.
Park J, Koito H, Li J, Han A (2009) Microfluidic compartmentalized co-culture platform for CNS axon myelination research. Biomedical Microdevices 11: 1145-53
Pellicanò A, Leone I, Imeneo M, Amorosi A, Luzza F (2008) Co-culture of human gastric endoscopic biopsies with Helicobacter pylori: A simple method for studying early phases of bacteria-host interaction. Journal of Microbiological Methods 75: 346-349.
Polk D B, Peek R M (2010) Helicobacter pylori: gastric cancer and beyond. Nature Reviews Cancer 10: 403-414.
Possemiers S, Verthé K, Uyttendaele S, Verstraete W (2004) PCR-DGGE-based quantification of stability of the microbial community in a simulator of the human intestinal microbial ecosystem. FEMS Microbiology Ecology 49: 495-507.
Qin J, Li R, Raes J, Arumugam M, Burgdorf K S, Manichanh C, Nielsen T, Pons N, Levenez F, Yamada T, Mende D R, Li J, Xu J, Li S, Li D, Cao J, Wang B, Liang H, Zheng H, Xie Y, Tap J, Lepage P, Bertalan M, Batto J-M, Hansen T, Le Paslier D, Linneberg A, Nielsen H B, Pelletier E, Renault P, Sicheritz-Ponten T, Turner K, Zhu H, Yu C, Li S, Jian M, Zhou Y, Li Y, Zhang X, Li S, Qin N, Yang H, Wang J, Brunak S, Dore J, Guarner F, Kristiansen K, Pedersen O, Parkhill J, Weissenbach J, Bork P, Ehrlich S D, Wang J (2010) A human gut microbial gene catalogue established by metagenomic sequencing. Nature 464: 59-65.
Rajilic-Stojanovic M, Maathuis A, Heilig H G H J, Venema K, de Vos W M, Smidt H (2010) Evaluating the microbial diversity of an in vitro model of the human large intestine by phylogenetic microarray analysis. Microbiology 156: 3270-3281.
Romano, M, Razandi, Sekhon, S, Krause, W J, Ivey, K J (1988) Human cell line for study of damage to gastric epithelial cells in vitro. Journal of Laboratory and Clinical Methods 111: 430-440.
Round J L, Mazmanian S K (2009) The gut microbiota shapes intestinal immune responses during health and disease. Nature Reviews Immunology 9: 313-323.
Saldarriaga Fernández I C, Busscher H J, Metzger S W, Grainger D W, van der Mei H C (2011) Competitive time- and density-dependent adhesion of Staphylococci and osteoblasts on crosslinked poly(ethylene glycol)-based polymer coatings in co-culture flow chambers. Biomaterials 32: 979-984.
Saleh-Lakha S, Trevors J T (2010) Perspective: Microfluidic applications in microbiology. Journal of Microbiological Methods 82: 108-111.
Sandek A, Rauchhaus M, Anker S D, von Haehling S (2008) The emerging role of the gut in chronic heart failure. Current Opinion in Clinical Nutrition and Metabolic Care 11:632-639.
Schloss P, Handelsman J (2005) Metagenomics for studying unculturable microorganisms: cutting the Gordian knot. Genome Biology 6: 229.
Sekiguchi M, Sakakibara K, Fujii G (1978) Establishment of cultured cell lines derived from a human gastric carcinoma. The Japanese Journal of Experimental Medicine 48: 61-68.
Sergent T, Ribonnet L, Kolosova A, Garsou S, Schaut A, De Saeger S, Van Peteghem C, Larondelle Y, Pussemier L, Schneider Y-J (2008) Molecular and cellular effects of food contaminants and secondary plant components and their plausible interactions at the intestinal level. Food and Chemical Toxicology 46: 813-841.
Shah P, Vedarethinam I, Kwasny D, Andresen L, Dimaki M, Skov S, Svendsen W E (2011) Microfluidic bioreactors for culture of non-adherent cells. Sensors and Actuator B. Chem. 156: 1002-1008
Shannon K M, Mutlu E A, Gillevet P M, Jaglin J A, Keshavarzian A (2010) Dysbiosis in Parkinson's Disease (PD)—Etiologic Factor?—A Pilot Study. American Neurological Association, American Neurological Association 135th Annual Meeting, Golden Gate Exhibit Hall of the San Francisco Marriott Marquis, San Francisco, USA, 13 Sep. 2010.
Shen H, Hu Y, Saltzman W M (2006) DNA diffusion in mucus: effect of size, topology of DNAs, and transfection reagents. Biophysical Journal 91: 639-644.
Skolimowski M, Nielsen M W, Emneus J, Molin S, Taboryski R, Sternberg C, Dufva M, Geschke O (2010) Microfluidic dissolved oxygen gradient generator biochip as a useful tool in bacterial biofilm studies. Lab on a Chip 10: 2162-2169.
Stappenbeck T S, Hooper L V, Gordon J I (2002) Developmental regulation of intestinal angiogenesis by indigenous microbes via Paneth cells. Proceedings of the National Academy of Sciences 99: 15451-15455.
Stybayeva, Gulnaz, He Z H U, Ramanculov, Erlan, Dandekar, Satya, George, Michael, Revzin, Alexander (2009) Micropatterned co-cultures of T-lymphocytes and epithelial cells as a model of mucosal immune system. Biochemical and Biophysical Research Communications 380: 575-580.
Subbiandoss G, Kuijer R, Grijpma D W, van der Mei H C, Busscher H J (2009) Microbial biofilm growth vs. tissue integration: “the race for the surface” experimentally studied. Acta Biomaterialia 5: 1399-1404.
Taff B M, Desai S P, Voldman J (2007) Dielectrophoretically switchable microfluidic weir structures for exclusion-based single-cell manipulation. Proceedings of the 11th International Conference on Micro Total Analysis Systems (micro-TAS 2007) 8-10
Tumarkin E, Tzadu L, Csaszar E, Seo M, Zhang H, Lee A, Peerani R, Purpura K, Zandstra P, Kumacheva E (2011) High-throughput combinatorial cell co-culture using microfluidics. Integrative Biology 3: 653-62
Turnbaugh P J, Ley R E, Mahowald M A, Magrini V, Mardis E R, Gordon J I (2006) An obesity-associated gut microbiome with increased capacity for energy harvest. Nature 444: 1027-1131.
van den Abbeele P, Grootaert C, Marzorati M, Possemiers S, Verstraete W, Gerard P, Rabot S, Bruneau A, El Aidy S, Derrien M, Zoetendal E, Kleerebezem M, Smidt H, Van de Wiele T (2010) Microbial community development in a dynamic gut model is reproducible, colon region specific, and selective for Bacteroidetes and Clostridium Cluster IX. Applied and Environmental Microbiology 76: 5237-5246.
vanDuynhoven J, Vaughan E E, Jacobs D M, A. KempermanRr, van Velzen E J J, Gross G, Roger L C, Possemiers S, Smilde A K, Doren, Westerhuis J A, Van de Wiele T (2011) Metabolic fate of polyphenols in the human superorganism. Proceedings of the National Academy of Sciences 108: 4531-4538.
van Nuenen M H M C, Diederick Meyer P, Venema K (2003) The effect of various inulins and Clostridium difficile on the metabolic activity of the human colonic microbiota in vitro. Microbial Ecology in Health and Disease 15: 137-144.
Vesterlund S, Karp M, Salminen S, Ouwehand A C (2006) Staphylococcus aureus adheres to human intestinal mucus but can be displaced by certain lactic acid bacteria. Microbiology 152: 1819-1826.
Vrieze A, Holleman F, Zoetendal E, de Vos W, Hoekstra J, Nieuwdorp M (2010) The environment within: how gut microbiota may influence metabolism and body composition. Diabetologia 53: 606-613.
Wang Z, Klipfell E, Bennett B J, Koeth R, Levison B S, DuGar B, Feldstein A E, Britt E B, Fu X, Chung Y-M, Wu Y, Schauer P, Smith J D, Allayee H, Tang W H W, DiDonato J A, Lusis A J, Hazen S L (2011) Gut flora metabolism of phosphatidylcholine promotes cardiovascular disease. Nature 472: 57-63.
Wen L, Ley R E, Volchkov P Y, Stranges P B, Avanesyan L, Stonebraker A C, Hu C, Wong F S, Szot G L, Bluestone J A, Gordon J I, Chervonsky A V (2008) Innate immunity and intestinal microbiota in the development of Type 1 diabetes. Nature 455: 1109-1113.
Whitesides G M (2006) The origins and the future of microfluidics. Nature 442: 368-373.
Wilmes P (2011) Microbial Community Proteomics. Handbook of Molecular Microbial Ecology I, ed. de Bruijn F J, pp. 627-635. John Wiley and Sons Inc., Hoboken, N.J., USA.
Yamashita S, Konishi K, Yamazaki Y, Taki Y, Sakane T, Sezaki H, Furuyama Y (2002) New and better protocols for a short-term Caco-2 cell culture system. Journal of Pharmaceutical Sciences. 91: 669-79
Zheng W, Wang Z, Zhang W and Jiang X (2010) A simple PDMS-based microfluidic channel design that removes bubbles for long-term on-chip culture of mammalian cells. Lab Chip. 10: 2906-10

1. Cell culture apparatus comprising at least two adjacent cell cultivation channels separated by a permeable or semipermeable membrane, wherein at least one channel has a cross section for a majority of its length that has two dimensions, and wherein at least one dimension does not exceed 500 μm, said channel being provided with entrance and exit means to permit the passage of media through at least a portion of the channel having a cross sectional area of no more than 1 mm².

2. Apparatus according to claim 1, wherein said second dimension is between 100 nm and 5 mm, and is preferably no more than 2 mm.

3. Cell culture apparatus comprising at least two adjacent cell cultivation channels separated by a permeable or semipermeable membrane, wherein at least one channel, for the majority of its length, has a cross sectional area of no more than 1 mm², said channel being provided with entrance and exit means to permit the passage of media through at least a portion of the channel having a cross sectional area of no more than 1 mm².

4. Apparatus according to any preceding claim, made of plastic, preferably polycarbonate or polystyrene.

5. Apparatus according to any preceding claim, wherein said apparatus is constructed in layers, with individual layers for each channel and for each membrane.

6. Apparatus according to any preceding claim, wherein the membrane does not permit passage of cells from one channel into another channel.

7. Apparatus according to claim 6, wherein the membrane is semipermeable.

8. Apparatus according to any preceding claim, wherein each cell culture channel separated by a membrane has a majority of its length with a cross sectional area of no more than 1 mm².

9. Apparatus according to any preceding claim, wherein each channel has a uniform cross section for substantially its entire length between entrance and exit means.

10. Apparatus according to any preceding claim, wherein the adjacent channels take the form of a paired helix.

11. Apparatus according to any preceding claim, comprising three or more channels.

12. Apparatus according to claim 11, wherein at least two channels are cell culture channels and the third is a perfusion channel.

13. Apparatus according to claim 12, wherein the perfusion channel is separated from one cell culture channel by a permeable or semipermeable membrane.

14. Apparatus according to any preceding claim, further comprising means to monitor growth of cell cultures and/or molecular interactions between cell cultures when present in said cell cultivation channels, preferably wherein said means allow the interrogation of molecular interactions by molecular techniques for generating information about at least a qualitative and/or quantitative attribute of said interactions.

15. Apparatus according to claim 14, wherein said techniques comprise imaging and/or spectroscopic techniques and/or any combination thereof, such as optical imaging, and preferably including fluorescence microscopy, and/or infrared-spectroscopy.

16. Apparatus according to claim 14 or 15, wherein said molecular techniques comprise one or more of genomics, proteomics, metabolomics, transcriptomics, or other molecular analysis techniques.

17. A method for making apparatus according to any preceding claim comprising constructing the apparatus in layers and sandwiching a suitable membrane between layers defining adjacent channels.

18. A method according to claim 17, wherein two channel-containing layers are provided, each having a channel provided in one surface, a groove in the surface defining said channel, leaving one side open, such that when the channel-containing sides of the layers are brought together, they can be brought together such that the channels are in register, and locating a membrane-containing layer between the two channel-containing layers, thereby to locate a membrane between the two channels, and securing the layers together, the membrane defining the final side of each channel.

19. A method according to claim 18, wherein one or more further, channel-containing layers are provided between the two said channel-containing layers, said further layers having one or more cut-outs defining said channels, and wherein membrane-containing layers separate each channel-containing layer.

20. A method for modelling interaction between two or more cell cultures, comprising establishing and monitoring said cultures separately in cell cultivation channels of apparatus according to any of claims 1 to 16.

21. A method according to claim 20, wherein nutrient media for at least one cell culture is supplied via a perfusion channel provided adjacent the cell cultivation channel and separated therefrom by a permeable or semipermeable membrane.

22. A method according to claim 20 or 21, wherein nutrient media for at least one cell culture is supplied via the entrance and exit means of the cell cultivation channel.

23. A method according to any of claims 20 to 22, wherein one cell culture is a mammalian, preferably human, cell lines, such as Caco-2 co-cultured with HT29-MTX or tissue, and the other cell culture is a microbial colony, such as a consortium, especially a biofilm.

24. A method according to any of claims 20 to 23, wherein the cell cultures include at least first and second cell cultures, and wherein said first cell culture is pathogenic to said second cell culture.

25. A method according to any of claims 20 to 24, wherein the cell cultures include at least first and second cell cultures, and wherein said first cell culture is aerobic and said second cell culture is anaerobic.

26. A method according to any of claims 20 to 25, wherein interactions between said cell cultures are monitored by monitoring means.

27. A method according to any of claims 20 to 26, wherein oxygen levels in at least one cell culture or perfusion channel are monitored by dissolved oxygen concentration monitoring means.

28. A method according to any of claims 20 to 27, comprising a plurality of apparatus according to any of claims 1 to 16 fluidically connected in series, optionally with each said apparatus having the same or different cell cultures and/or nutrient media supplies.
